(19) United States
(12) Patent Application Publication
(10) Pub. No.: US 2020/0101041 A1
(43) Pub. Date: Apr. 2, 2020

(54) METHODS AND COMPOSITIONS FOR ENHANCING HEALTH

(71) Applicant: Ojai Energetics PBC, Ojai, CA (US)

(72) Inventor: William Kleidon, Ojai, CA (US)

(21) Appl. No.: 16/585,630

(22) Filed: Sep. 27, 2019

Related U.S. Application Data
(63) Continuation of application No. PCT/US18/24061, filed on Mar. 23, 2018.
(60) Provisional application No. 62/479,091, filed on Mar. 30, 2017.
(60) Provisional application No. 62/506,475, filed on May 15, 2017.
(60) Provisional application No. 62/632,965, filed on Feb. 20, 2018.

(51) Int. Cl. A61K 31/352; A61K 31/01; A61K 31/015; A61K 31/045

(52) U.S. Cl. CPC A61K 31/352; A61K 31/045; A61K 31/015; A61K 31/01

(57) ABSTRACT

Compositions, methods, and kits described herein may be formulated as a nutritional supplement or a dietary supplement. A composition described herein may provide nutrients or compounds that may otherwise not be consumed in sufficient quantities in a normal diet, and may contribute to physical wellbeing and emotional wellbeing of a subject.

### CROSS-REFERENCE

This application claims priority to International Patent Application Serial No. PCT/US18/24061, filed Mar. 23, 2018, which claims the benefit of U.S. Provisional Patent Application Ser. No. 62/479,091, filed Mar. 30, 2017, U.S. Provisional Patent Application Ser. No. 62/506,475, filed May 15, 2017, and U.S. Provisional Patent Application Ser. No. 62/632,965, filed Feb. 20, 2018, each of which is entirely incorporated herein by reference.

### BACKGROUND

Terpenoids are compounds that may have beneficial effects on a subject when taken as a nutritional supplement or a dietary supplement. Terpenoids may possess benefits to the health, physical wellbeing, and/or emotional wellbeing of a subject.

Terpenoids may be used to treat or decrease symptoms of a number of classes of disorders, such as anti-inflammatory disorders, psychiatric disorders, and sleep disorders. Terpenoid degradation compounds may be formed as a by-product during the manufacture of a composition comprising terpenoids. Terpenoid degradation compounds may have little benefit, no effect, or harmful effects on a subject.

### SUMMARY

In one aspect, the present disclosure provides a unit dose comprising: (i) a mixture of carboxylated cannabinoids and decarboxylated cannabinoids, and (ii) one or more terpenoids, wherein a wt/wt ratio of decarboxylated cannabinoids to carboxylated cannabinoids is greater than 0.5, and wherein the unit dose is substantially free of terpenoid degradation compounds. In some embodiments, the unit dose comprises at least 5 mg of decarboxylated cannabinoids. In some embodiments, decarboxylated cannabinoids comprises Δ⁹ tetrahydrocannabinol.

In some embodiments, one or more terpenoids is selected from the group consisting of: myrcene, limonene, linalool, trans-ocimene, beta-pinene, alpha-pinene, beta-caryophyllene, delta-3-carene, trans-gamme-bisabolene, trans-alpha-farnesene, beta-fenchol, alpha-humulene, and guajol. In some embodiments, one or more terpenoid degradation compounds is selected from the group consisting of: geraniol, geranyl isobutyrate, p-cymenene, p-cymene, p-mentha-1,5,8-triene, carvone, 3-methyl-6-(1-methylethylidene)-2-cyclohexen-1-one, 3-methyl-6-(1-methylethenyl)-2-cyclohexen-1-one, eucarvone, thymol, p-mentha-1(7),8-dien-2-ol, perillyl alcohol, camphene, beta-myrcene, alpha-phellandrene, alpha-terpinene, gamma-terpinene, terpinolene, 4-hydroxy-2-methyl-2-cyclohexenone, p-cymenene, o-cymene, 3-caren-2-one, 3-caren-5-one, 3-carene oxide, 3-carene-2,5-dione, trans-2-hydroxy-3-caren-5-one, thymol, carvacrol, 1,4-cineole, eucalyptol, 3-(1-methylethyl)-6-oxo-2-heptenal, and 3,7-dimethyl6-oxo-2-octenal. In some embodiments, the unit dose further comprises a trace amount of an acid. In some embodiments, the unit dose further comprises a pharmaceutically acceptable excipient.

In some embodiments, the pharmaceutically acceptable excipient is selected from the group consisting of: a binder, a filler, a plasticizer, a lubricant, an anti-foaming agent, a buffering agent, a polymer, a antioxidant, a preservative, a chelating agent, a flavorant, a colorant, an odorant, a suspending agent, and a combination thereof. In some embodiments, the unit dose is formulated for oral, topical, inhalation, intravenous, or intramuscular administration. In some embodiments, the unit dose is in a solid form. In some embodiments, the unit dose is in a liquid form. In some embodiments, the unit dose is a tablet, a chewable tablet, a capsule, a caplet, a pill, a granule, an emulsion, a gel, a spray, a plurality of beads encapsulated in a capsule, a powder, a suspension, a liquid, a semi-liquid, a semi-solid, a solution, a syrup, or a slurry. In some embodiments, the unit dose retains at least 80% of the cannabinoids after placement in a sealed container for 6 months at a temperature of about 25° C. and a relative humidity level of about 50%. In some embodiments, the unit dose is packaged into a container selected from the group consisting of a tube, ajar, a box, a vial, a bag, a tray, a drum, a bottle, a syringe, and a can. In some embodiments, a kit comprises a unit dose disclosed herein and instructions for supplementing the mixture of carboxylated cannabinoids and decarboxylated cannabinoids and one or more terpenoids to a subject in need thereof.

In an aspect, the present disclosure provides a kit for preparing Δ⁹ tetrahydrocannabinol comprising: (i) an acid present in an amount effective for conversion of at least 50% of tetrahydrocannabinolic acid to the Δ⁹ tetrahydrocannabinol, (ii) a reaction vessel configured to hold a reaction mixture comprising the acid and the tetrahydrocannabinolic acid, and (iii) instructions for performing the conversion utilizing the acid. In some embodiments, the kit further comprises tetrahydrocannabinolic acid. In some embodiments, the acid is a weak acid. In some embodiments, the acid has a pKa from about 3 to about 7.

In some embodiments, the acid is selected from the group consisting of: lactic acid, citric acid, malic acid, acetic acid, benzoic acid, ascorbic acid, tartric acid, and oxalic acid.

In an aspect, the present disclosure provides a method of supplementing one or more cannabinoids and one or more terpenoids to a subject in need thereof, the method comprising administering to the subject a unit dose comprising: a mixture of carboxylated cannabinoids and decarboxylated cannabinoids, and one or more terpenoids, wherein a wt/wt ratio of decarboxylated cannabinoids to carboxylated cannabinoids is greater than 0.5, and wherein the unit dose is substantially free of terpenoid degradation compounds. In some embodiments, the subject suffers from a symptom selected from the group consisting of: pain, stress, nausea, vomiting, sleeplessness, anxiety, and appetite loss. In some embodiments, the unit dose is administered orally, topically, by inhalation, intravenously, or intramuscularly. In some embodiments, the unit dose is administered at least once per day. In some embodiments, the method further comprises monitoring a health state or condition of the subject subsequent to administering the unit dose to the subject.

In an aspect, the present disclosure provides a method of supplementing one or more cannabinoids and one or more terpenoids to a subject in need thereof, the method comprising administering to the subject a unit dose disclosed herein. In some embodiments, the subject suffers from a symptom selected from the group consisting of: pain, stress, nausea, vomiting, sleeplessness, anxiety, and appetite loss. In some embodiments, the unit dose is administered orally, topically, by inhalation, intravenously, or intramuscularly. In some embodiments, the unit dose is administered at least once per day. A unit dose may be administered intravenously to a subject prior to, during, or after a surgical procedure (e.g., within 1 minute, 10 minutes, 20 minutes or 30 minutes after surgery).

In an aspect, the present disclosure provides a method of producing decarboxylated cannabinoids, comprising: (i) contacting a cannabis plant or a portion thereof with an acid to form a reaction mixture under conditions effective for converting carboxylated cannabinoids present in the cannabis plant to decarboxylated cannabinoids; and (ii) separating the cannabis plant or a portion thereof from the decarboxylated cannabinoids, thereby producing the decarboxylated cannabinoids. In some embodiments, the decarboxylated cannabinoids comprise Δ⁹ tetrahydrocannabinol. In some embodiments, a wt/wt ratio of decarboxylated cannabinoids to carboxylated cannabinoids in the reaction mixture of (ii) is greater than 0.1. In some embodiments, the conditions are at a temperature of less than 300° C. In some embodiments, external heating is not applied during the converting of the carboxylated cannabinoids to the decarboxylated cannabinoids. In some embodiments, the acid is a weak acid. In some embodiments, the contacting comprises blending, mixing, stirring, or a combination thereof. In some embodiments, the separating is selected from the group consisting of: filtration, extraction, centrifugation, solubilization, concentration, washing, electrolysis, adsorption, purification, chromatography, fractionation, crystallization, and a combination thereof.

In another aspect, the present disclosure provides a mixture comprising: (i) carboxylated cannabinoids and decarboxylated cannabinoids, (ii) one or more terpenoids, and (iii) an acid, wherein a wt/wt ratio of decarboxylated cannabinoids to carboxylated cannabinoids is greater than 0.1, and wherein the acid is present in an amount effective in converting at least a portion of carboxylated cannabinoids to decarboxylated cannabinoids. In some embodiments, the carboxylated cannabinoids comprise tetrahydrocannabinolic acid. In some embodiments, the mixture comprises at least 0.05 mol of the decarboxylated cannabinoids. In some embodiments, the decarboxylated cannabinoids comprise Δ⁹ tetrahydrocannabinol. In some embodiments, a wt/wt ratio of Δ⁹ tetrahydrocannabinol to tetrahydrocannabinolic acid is greater than about 0.1. In some embodiments, the mixture is substantially free of terpenoid degradation compounds. In some embodiments, the acid is an organic acid.

In an aspect, the present disclosure provides a reaction vessel comprising a mixture disclosed herein, wherein the reaction vessel is configured to provide production of at least 10 g of the decarboxylated cannabinoids.

In an aspect, the present disclosure provides a method for generating a decarboxylated cannabinoid formulation, comprising: providing a reaction vessel comprising a mixture, wherein the mixture comprises: carboxylated cannabinoids and decarboxylated cannabinoids, one or more terpenoids, and an acid, wherein a wt/wt ratio of decarboxylated cannabinoids to carboxylated cannabinoids is greater than 0.1, and wherein the acid is present in an amount effective in converting at least a portion of carboxylated cannabinoids to decarboxylated cannabinoids; and mixing the mixture to yield the decarboxylated cannabinoid formulation. In some embodiments, the decarboxylated cannabinoid formulation comprises at least 5 mg of decarboxylated cannabinoids. In some embodiments, the decarboxylated cannabinoid formulation comprises Δ⁹ tetrahydrocannabinol. In some embodiments, the one or more terpenoids is selected from the group consisting of: myrcene, limonene, linalool, trans-ocimene, beta-pinene, alpha-pinene, beta-caryophyllene, delta-3-carene, trans-gamme-bisabolene, trans-alpha-farnesene, beta-fenchol, alpha-humulene, and guajol. In some embodiments, the acid is a weak acid. In some embodiments, the acid has a pKa from about 3 to about 7. In some embodiments, the acid is selected from the group consisting of: lactic acid, citric acid, malic acid, acetic acid, benzoic acid, ascorbic acid, tartric acid, and oxalic acid.

In an aspect, the present disclosure provides a unit dose comprising: a mixture of carboxylated cannabinoids and decarboxylated cannabinoids, and one or more terpenoids, wherein a wt/wt ratio of decarboxylated cannabinoids to carboxylated cannabinoids is greater than 0.5, wherein the unit dose is substantially free of terpenoid degradation compounds, and wherein the unit dose is substantially free of an acid. In some embodiments, the acid is a weak acid. In some embodiments, the acid has a pKa from about 3 to about 7. In some embodiments, the acid is selected from the group consisting of: lactic acid, citric acid, malic acid, acetic acid, benzoic acid, ascorbic acid, tartric acid, and oxalic acid.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “about,” as used herein, generally refers to an acceptable error range for the particular value as determined by one of ordinary skill in the art, which may depend in part on how the value is measured or determined. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, and within 2-fold, of a value.

The term “subject,” as used herein, generally refers to an animal. The subject may have or be suspected of having a disease or ailment. A subject may be a mammal. Non-limiting examples of mammals include humans and animals, such as mice, monkeys, dogs and cats, including transgenic and non-transgenic mice. The methods described herein can be useful in both human therapeutics, pre-clinical, and veterinary applications. The subject may be a mammal. The subject may be human. Other mammals include, but are not limited to, apes, chimpanzees, orangutans, monkeys; domesticated animals (pets) such as dogs, cats, guinea pigs, hamsters, mice, rats, rabbits, and ferrets; domesticated farm animals such as cows, buffalo, bison, horses, donkey, swine, sheep, and goats; or exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, pandas, giant pandas, hyena, seals, sea lions, and elephant seals.

The term “administer,” as used herein, generally refers to providing a composition to a subject via a route of administration, including but not limited to intravenous, intraarterial, oral, parenteral, buccal, topical, transdermal, rectal, intramuscular, subcutaneous, intraosseous, transmucosal, or intraperitoneal routes of administration. A composition may be administered via a suppository, such as a vaginal or anal suppository. Oral routes of administration may be used. A unit dose may be administered via inhalation.

The term “effective amount” or “therapeutically effective amount,” as used herein, generally refers to an amount of a compound described herein that is sufficient to affect an intended, predetermined or prescribed application, including but not limited to, disease or condition treatment. The therapeutically effective amount can vary depending upon the application (e.g., in vitro or in vivo), or the subject and disease condition being treated, e.g., the weight and age of the subject, the severity of the disease condition and the manner of administration. The term also may apply to a dose that induces a particular response in target cells, e.g., reduction of proliferation or down regulation of activity of a target protein. The specific dose may vary depending on the particular compounds chosen, the dosing regimen to be followed, whether it is administered in combination with other compounds, timing of administration, the tissue to which it is administered, and the physical delivery system in which it is carried.

The term “isolated,” as used herein, generally refers to a preparation of a substance devoid of at least some of the other components that can also be present where the substance or a similar substance naturally occurs or is initially obtained from. Thus, for example, an isolated substance can be prepared by using a purification technique to enrich it from a source mixture. Enrichment can be measured on an absolute basis, such as weight per volume of solution, or it can be measured in relation to a second, potentially interfering substance present in the source mixture. Increasing enrichment may be used. A substance can also be provided in an isolated state by a process of artificial assembly, such as by chemical synthesis.

The term “substantially free,” as used herein, generally refers to a composition that has less than about 25% (e.g., by weight), less than about 15%, less than about 10%, less than about 5%, less than about 1%, less than about 0.5%, less than 0.1% or even less of a specified component. Such composition may not have a detectable amount of such specified component. For example a composition that is substantially free of a weak acid (e.g., an acid with a pKa of at most about 10) can have less than about 1% of the weak acid. The percentage can be determined as a percent of the total composition or a percent of a subset of the composition. For example, a composition that is substantially free of a weak acid can have less than 1% of the weak acid as a percent of the total composition, or as a percent of the acids in the composition. The percentages can be mass, molar, or volume percentages. The presence or concentration of such component may be determined spectroscopically, such as chromatography or nuclear magnetic resonance.

The term “synergistic,” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 50%, 75%, 100%, 110%, 120%, 150%, 200%, 250%, 350%, or 500% or more than the effect on a subject with one of the components alone, or the additive effects of each of the components when administered individually. The effect can be any of the measurable effects described herein.

The term “cannabis plant,” as used herein, generally refers to a plant that is part of a genus of a flowering plant in the family Cannabaceae, and may include three species or subspecies: sativa, indica, and ruderalis. A cannabis plant may comprise a number of different parts, including a node, a plant stem, a fan leaf, and a flower. The flower of a cannabis plant may be a male or a female flower. The female flower may comprise a flower, a pistil, a cola, a trichome, and a calyx.

The term “cannabinoid,” as used herein, generally refers to a cannabinoid compound that has been isolated or identified in a cannabis plant. A cannabinoid compound may act on a cannabinoid receptor in a cell. The cannabinoid may alter physiological processes, including altering neurotransmitter release in the brain, appetite, pain-sensation, mood, and memory. A cannabinoid compound may have a C₂₁ terpenophenolic core.

The term “carboxylated cannabinoid,” as used herein, generally refers to a compound that has been isolated or identified in a cannabis plant and possesses a carboxylic acid moiety (i.e. —COOH). A carboxylated cannabinoid may be tetrahydrocannabinolic acid.

The term “decarboxylated cannabinoid,” as used herein, generally refers to a cannabinoid compound that previously possessed a carboxylic acid moiety (e.g. a carboxylated cannabinoid), and underwent a chemical reaction so to no longer possesses the carboxylic acid moiety. A decarboxylated cannabinoid may be a natural compound and may be present in a cannabis plant. A decarboxylated cannabinoid may be synthesized or produced via synthetic methods. A decarboxylated cannabinoid may be Δ⁹ tetrahydrocannabinol.

The term “terpenoid,” as used herein, generally refers to an organic compound that is composed of isoprene units, wherein each isoprene unit has the formula C₅H₈. The isoprene units may be connected via covalent bonds. Terpenoids may have the formula (C₅H₈)ₙ, wherein n is an integer of 1 or more, such as 2, 3, 4, 5, or more. A terpenoid may be a monoterpenoid (C₁₀ skeleton), sesquiterpenoid (Cis skeleton), diterpenoid (C₂₀ skeleton), or treterpenoid (C₃₀ skeleton). Terpenoids may possess beneficial effects on a subject, and may be used as a dietary supplement or nutritional supplement.

The term “terpenoid degredation product,” as used herein, generally refers to an organic compound that is a product of a reaction performed on a terpenoid. A terpenoid may degrade into multiple fragments, wherein each fragment may be considered as a terpenoid degradation product. A terpenoid degradation product may be formed during a reaction such as heating, burning, or smoking a terpenoid compound or a composition comprising a terpenoid. A terpenoid degradation product may be formed by application of heat of at least about 50° C., 75° C., 100° C., 200° C., 300° C., 400° C., 500° C., 600° C., 700° C., 800° C., 900° C., 1000° C., or more. A terpenoid degradation product may not possess the beneficial properties of the terpenoid from which it was derived.

Cannabinoid compounds can be divided into ten subclasses. Subclasses of cannabinoid compounds include the cannabigerol class, cannabichromene class, cannabidiol class, delta-9-tetrahydrocannabinol class, delta-8-tetrahydrocannabinol class, cannabicyclol class, cannabielsoin class, cannabinol and cannabinodiol class, cannabitriol class, and a miscellaneous cannabinoids class.

Non-limiting examples of cannabinoid compounds in the cannabigerol class include cannabigerolic acid (CBGA), cannabigerolic acid monomethylether (CBGAM), cannabigerol (CBG), cannabigerol monomethylether (CBGM), cannabigerovarinic acid (CBGVA), and cannabigerovarin (CBGV).

Non-limiting examples of cannabinoid compounds in the cannabichromene class include cannabichromenic acid (CBCA), cannabichromene (CBC), cannabichromevarinic acid (CBCVA), and cannabichromevarin (CBCV).

Non-limiting examples of cannabinoid compounds in the cannabidiol class include cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), and cannabidiorcol (CBD-C₁).

Non-limiting examples of cannabinoid compounds in the delta-9-tetrahydrocannabinol class include delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THC-C₄), dena-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), and delta-7-cis-iso-tetrahydrocannabivarin.

Non-limiting examples of cannabinoid compounds in the delta-8-tetrahydrocannabinol class include delta-8-tetrahydrocannabinolic acid (As-THCA), and delta-8-tetrahydrocannabinol (Δ⁸-THC).

Non-limiting examples of cannabinoid compounds in the cannabicyclol class include cannabicyclolic acid (CBLA), cannabicyclol (CBL), and cannabicyclovarin (CBLV).

Non-limiting examples of cannabinoid compounds in the cannabielsoin class include cannabielsoic acid A (CBEA-A), cannabielsoic acid B (CBEA-B), and cannabielsoin (CBE).

Non-limiting examples of cannabinoid compounds in the cannabinol and cannabinodiol class include cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), and cannabinodivarin (CBVD).

Non-limiting examples of cannabinoid compounds in the cannabitriol class include cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), and ethoxy-cannabitriolvarin (CBTVE).

Non-limiting examples of cannabinoid compounds in the miscellaneous cannabinoids class, include dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

Terpenoid compounds that have been isolated from the essential oil of a cannabis plant may include myrcene, limonene, linalool, trans-ocimene, beta-pinene, alpha-pinene, beta-caryophyllene, delta-3-carene, trans-gamma-bisabolene, trans-alpha-farnesene, beta-fenchol, beta-phellandrene, alpha-humulene (alpha-caryophyllene), guajol, alpha-guaiene, alpha-eudesmol, terpinolene, alpha-selinene, alpha-terpineol, fenchone, camphene, cis-sabinene hydrate, cis-ocimene, beta-eudesmol, beta-selinene, alpha-trans-bergamotene, gamma-eudesmol, borneol, cis-beta-farnesene, gamma-curcumene, cis-gamma-bisabolene, alpha-thujene, epi-alpha-bisabolol, ipsdienol, alpha-ylangene, beta-elemene, alpha-cis-bergamotene, gamma-muurolene, alpha-cadinene, alpha-longipinene, and caryophyllene oxide.

Nitrogen-containing compounds have been isolated from a cannabis plant. Spermidine-type alkaloids that have been isolated from Cannabis sativa may include cannabisativine and anhydrocannabisativine. Other nitrogen-containing compounds that have been isolated from a cannabis plant include, but is not limited to, n-trans-feruloyityramine, n-p-coumaroyltyramine, n-trans-caffeoyltyramine, grossamide, cannabisin-A, cannabisin-B, cannabisin-C, and cannabisin-D.

Flavonoids are compounds that may be plant or fungus secondary metabolites. Generally, flavonoids have a C₁₅ skeleton. Flavonoids have been identified in a cannabis plant, including apigenin, luteolin, kaempferol, quercetin, orientin, vitexin, cannflavin A, and cannflavin B.

Additional compounds that have been isolated from a cannabis plant include unsaturated fatty acids and noncannabinoid phenols, including, but not limited to, linoleic acid, alpha-linolenic acid, oleic acid, cannabispiran, isocannabispiran, cannabistilbene-I, cannabistilbene-II, cannithrene-1, and cannithrene-2.

### Compositions

The present disclosure provides a unit dose of a composition that may comprise a mixture of carboxylated cannabinoids and decarboxylated cannabinoids and one or more terpenoids. The composition may comprise decarboxylated cannabinoids (e.g. Δ⁹ tetrahydrocannabinol) and carboxylated cannabinoids (e.g. tetrahydrocannabinolic acid).

The composition may comprise a ratio of decarboxylated cannabinoids to carboxylated cannabinoids of at least about 0.01:1, 0.05:1, 0.1:1, 0.5:1, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, or more. The ratio of decarboxylated cannabinoids to carboxylated cannabinoids may also be described as a single value of at least about 0.01, 0.05, 0.1, 0.5, 1, 2, 3, 4, 5, 6, 7, 8, 9, 10, 15, 20, 30, 40, 50, 60, 70, 80, 90, 100, or more. The ratio of decarboxylated cannabinoids to carboxylated cannabinoids in a composition of the current disclosure or a unit dose may be different than the ratio of the products isolated directly from a natural source. For example, a ratio of decarboxylated cannabinoid (e.g. Δ⁹ tetrahydrocannabinol) to carboxylated cannabinoid (e.g. tetrahydrocannabinolic acid) may be 0.05:1 when isolated from a natural source, such as a cannabis plant. After undergoing a method described herein, the ratio of decarboxylated cannabinoid (e.g. Δ⁹ tetrahydrocannabinol) to carboxylated cannabinoid (e.g. tetrahydrocannabinolic acid) may be 1:1, 2:1, 5:1, or higher.

Any of the components described herein, including Δ⁹ tetrahydrocannabinol, may be used in a composition in free form, isolated form, purified from a natural source, and/or purified or prepared from a synthetic source. The natural source can be an animal source or plant source. The components can be pure to at least about 95, 97, 99, 99.5, 99.9, 99.99, or 99.999%.

A dose of the present disclosure, which can be a unit dose, can comprise more than about 1 milligram (mg), 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of a cannabinoid compound. A dose of a composition of the present disclosure, which can be a unit dose, can comprise about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of a cannabinoid compound. A dose of a composition of the present disclosure, which can be a unit dose, can comprise less than about 1 mg 2 mg 3 mg 4 mg, 5 mg 6 mg, 7 mg, 8 mg 9 mg, 10 mg 11 mg 12 mg, 13 mg 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of a cannabinoid compound.

A unit dose of Δ⁹ tetrahydrocannabinol can be at least about 10 mg, 20 mg, 30 mg, 40 mg 50 mg 60 mg 70 mg, 80 mg 90 mg, 100 mg, 150 mg 200 mg, 250 mg, 300 mg 350 mg, 400 mg 450 mg 500 mg, 550 mg, 600 mg 650 mg 700 mg, 750 mg 800 mg 850 mg, 900 mg 950 mg, or 1000 mg. A unit dose of Δ⁹ tetrahydrocannabinol can be from about 1 mg to about 20 mg, from 3 mg to about 15, from 5 mg to about 10 mg. A dose of Δ⁹ tetrahydrocannabinol can comprise at least about 0.001 moles (mol) Δ⁹ tetrahydrocannabinol, 0.005 mol, 0.01 mol, 0.015 mol, 0.02 mol, 0.03 mol, 0.04 mol, 0.05 mol, 0.06 mol, 0.07 mol, 0.08 mol, 0.09 mol, 0.1 mol, 0.2 mol, 0.3 mol, 0.4 mol, 0.5 mol, or more.

A unit dose may comprise at least about 10 milligram (mg), 20 mg 30 mg, 40 mg 50 mg 60 mg 70 mg 80 mg, 90 mg 100 mg, 150 mg, 200 mg 250 mg, 300 mg 350 mg, 400 mg, 450 mg 500 mg 550 mg, 600 mg, 650 mg 700 mg 750 mg, 800 mg 850 mg 900 mg, 950 mg or 1000 mg of tetrahydrocannabinolic acid.

A unit dose may comprise at least about 10 mg 20 mg, 30 mg 40 mg 50 mg, 60 mg 70 mg 80 mg 90 mg 100 mg, 150 mg 200 mg 250 mg, 300 mg 350 mg 400 mg, 450 mg 500 mg 550 mg, 600 mg, 650 mg, 700 mg, 750 mg, 800 mg, 850 mg, 900 mg, 950 mg, or 1000 mg of a terpenoid.

A unit dose may be substantially free of a terpenoid degradation compound. A unit dose may comprise less than about 1000 mg, 900 mg, 800 mg, 700 mg, 600 mg, 500 mg, 400 mg, 300 mg, 200 mg, 100 mg, 50 mg, 40 mg, 30 mg, 20 mg, or 10 mg of a terpenoid degradation compound. A unit dose may comprise less than about 1000 mg, 900 mg 800 mg, 700 mg 600 mg, 500 mg, 400 mg, 300 mg, 200 mg, 100 mg, 50 mg, 40 mg, 30 mg, 20 mg, or 10 mg of multiple terpenoid degradation compounds. A unit dose may comprise less than about 10%, 5%, 4%, 3%, 2%, 1%, 0.1%, or 0.01% of a terpenoid degradation compound by weight. A unit dose may comprise less than about 10%, 5%, 4%, 3%, 2%, 1%, 0.1%, or 0.01% of a terpenoid degradation compound by volume. A unit dose may comprise at most about 1000 mg, 900 mg, 800 mg, 700 mg, 600 mg, 500 mg, 400 mg, 300 mg, 200 mg, 100 mg, 50 mg, 40 mg, 30 mg, 20 mg, or 10 mg of a terpenoid degradation compound.

A unit dose may be substantially free of an acid, wherein the acid may be used to convert a carboxylated cannabinoid to a decarboxylated cannabinoid. A unit dose may comprise less than about 1000 mg, 900 mg, 800 mg, 700 mg, 600 mg, 500 mg, 400 mg 300 mg 200 mg, 100 mg, 50 mg, 40 mg, 30 mg, 20 mg, or 10 mg of an acid. A unit dose may comprise less than about 1000 mg, 900 mg, 800 mg, 700 mg, 600 mg, 500 mg, 400 mg, 300 mg, 200 mg, 100 mg, 50 mg 40 mg, 30 mg, 20 mg, or 10 mg of multiple acids. A unit dose may comprise less than about 10%, 5%, 4%, 3%, 2%, 1%, 0.1%, or 0.01% of an acid by weight. A unit dose may comprise less than about 10%, 5%, 4%, 3%, 2%, 1%, 0.1%, or 0.01% of an acid by volume. A unit dose may comprise at most about 1000 mg, 900 mg, 800 mg, 700 mg 600 mg, 500 mg, 400 mg 300 mg 200 mg, 100 mg, 50 mg, 40 mg, 30 mg, 20 mg, or 10 mg of an acid.

A unit dose of a composition of the current disclosure can result in a blood concentration, blood plasma concentration, or blood content of a compound in the composition certain amount after a given period of time. A unit dose of a composition may result in a blood content that may be measure from a sample of blood, a sample of blood plasma, a urine sample, a saliva swab, the subject's breath, or other samples of bodily fluids.

A unit dose of a cannabinoid can result in a blood concentration of the cannabinoid of at least about 0.5, 0.75, 1, 1.25, 1.5, 1.75, 2, 2.5, 3, 4, 5, 10, 20, 30, 40, 50, 60, 70, 80, 90, 100, 110, 120, 130, 140, 150, 160, 170, 180, 190, 200, 300, 400, 500 nanograms per milliliter (ng/mL) or greater. Alternatively, the unit dose of the cannabinoid can result in a blood concentration of the cannabinoid of at most about 500, 400, 300, 200, 190, 180, 170, 160, 150, 140, 130, 120, 110, 100, 90, 80, 70, 60, 50, 40, 30, 20, 10, 9, 8, 7, 6, 5, 4, 3, 2, 1, or 0.5 ng/mL. A unit dose of a cannabinoid can result in a blood concentration of the cannabinoid of about at least about 10, 20, 30, 40, 50, 60, 70, 80, 90, 100 ng/mL or higher. A unit dose of a cannabinoid can result in a blood concentration of the cannabinoid from 10 to 200 ng/mL, from 50 to 190 ng/mL, or from 90 to 170 ng/mL.

A daily dose of a cannabinoid can result in a blood concentration of the cannabinoid of at least about 0.5 ng/mL, 0.75 ng/mL, 1 ng/mL, 1.25 ng/mL, 1.5 ng/mL, 1.75 ng/mL, 2 ng/mL, 2.5 ng/mL, 3 ng/mL, 4 ng/mL, 5 ng/mL, 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 300 ng/mL, 400 ng/mL, 500 ng/mL or higher. A unit dose of a cannabinoid can result in a blood concentration of the cannabinoid from 10 to 200 ng/mL, from 50 to 190 ng/mL, or from 90 to 170 ng/mL.

Blood levels of a cannabinoid may peak about 1, 2, 3, 4, 5, 6, 7, 8, 9, 10, 12, 14, 16, 18, or 20 minutes after the first dose of a cannabinoid. Blood levels of a cannabinoid may remain detectable for about 1 minute, 2 minutes, 5 minutes, 10 minutes, 1 hour, 2 hours, 3 hours, 4 hours, 6 hours, 8 hours, 10 hours, 12 hours, 18 hours, 24 hours, or 36 hours after the last dose of the cannabinoid.

A composition of the current disclosure may be used in a combination therapy. A combination therapy may be administered by a combination treatment regimen. A combination treatment regimen may encompass treatment regimens in which administration of a compound described herein, or a pharmaceutically acceptable salt thereof, is initiated prior to, during, or after treatment with a second agent, and may continue until any time during treatment with the second agent or after termination of treatment with the second agent. The second agent being used in combination may be administered simultaneously or at different times and/or at decreasing or increasing intervals during the treatment period than the first agent.

A composition of the current disclosure may comprise two or more compounds. A composition may comprise two or more compounds of the current disclosure.

A composition of the current disclosure may be used in conjunction with an opioid. The opioid may act on an opioid receptor after administration to a subject. The combination of a composition of the current disclosure and an opioid may have a synergistic effect on a subject. The combination therapy may impart fewer and lessen the severity of side effects observed compared to when a compound or composition is administered as a single agent.

A composition of the current disclosure may be used in conjunction with a chemotherapy agent (e.g., paclitaxel, docetaxel, doxorubicin, bortezomib, gemcitabine, or cisplatin) or chemotherapy treatment. The chemotherapy agent or treatment may be radiation, hormonal therapy, targeted therapy, or a cytotoxic agent. When used in conjunction with chemotherapy treatment, the composition and chemotherapy treatment may exhibit synergistic effects.

A combination composition can be formulated to achieve a given, desired or predetermined molar ratio or mass ratio between two or more compounds of the composition. The molar ratio can be adjusted to account for the bioavailability, the uptake, and the metabolic processing of the one or more components of a combination composition. For example, if the bioavailability of a component is low, then the molar amount of a that component can be increased relative to other components in the combination composition. The circulating molar or mass ratio may be achieved within about 0.1, 0.5, 0.75, 1, 3, 5, or 10, 12, 24, or 48 hours after administration. The circulating molar or mass ratio can be maintained for a time period of about or greater than about 0.1, 1, 2, 5, 10, 12, 18, 24, 36, 48, 72, or 96 hours.

### Dosing Forms

The compositions described herein can be compounded into a variety of different dosage forms. It can be in an oral dosage form. The composition may be used orally, such as, for example, in the form of a tablet, a capsule, a pill, a granule, an emulsion, a gel, a plurality of beads encapsulated in a capsule, a powder, a suspension, a liquid, a semi-liquid, a semi-solid, a syrup, a slurry, a chewable form, caplets, soft gelatin capsules, lozenges or solution. Alternatively, a composition can be formulated for inhalation or for intravenous delivery. The compositions can also be formulated as a nasal spray or for injection when in solution form. Alternatively, the composition can be a liquid composition suitable for oral consumption.

A composition may also be formulated onto a solid or semi-solid support. The composition may be formulated on or in a polymeric material (e.g., silicone) and can be used as an injectable polymeric material (e.g., silicone) to prevent blood loss during traumatic injury or surgery. The polymeric material may be a biopolymer. The biopolymer may biodegradable or absorbable into the subject over a period of time.

The polymeric material may facilitate wound repair, assist in a decrease in perceived pain by the subject, exhibit antimicrobial properties, such as slowing the growth of microorganisms, or facilitate overall homeostatic balance in or around the wound or in the subject as a whole. The polymeric material may decrease shock, trauma, or oxidative stress to the area of or around the wound or in subject overall. The polymeric material may also be used as a vehicle for delivering therapeutic material. In some cases, a composition of the current disclosure and a second therapeutic material may be formulated onto a polymeric material that is use before, during, or after a surgical procedure or trauma.

The composition may be formulated for use during and after a surgical procedure, such as onto a medical device. The medical device may be a suture, a plug, thread, an implant, or a prosthetic. The composition may be formulated onto a material that is biodegradable or absorbable and may degrade within the body after at least about 1 day, 2 days, 3 days, 7 days, 1 month, 2 months, or more. Alternatively, the medical device (e.g. suture or plug) may be non-biodegradable or non-absorbable. The composition may facilitate a slow-release of compounds of the composition, which may be desired. The compositions can be formulated onto a medical device that is implanted into a subject during surgery, and may release one or more components over a time period of 1, 4, 6, 8, 12, 16, 20, 24, 36, 48 or more hours.

A composition described herein may be used to enhance the success or results of an implant or prosthetic procedure. For example, a composition may be administered before, during, or after an implant procedure. Implants may be used to replace a missing biological structure, support damaged structure, or enhance existing structure. In some embodiments, an implant may be subdermal. In some embodiments, an implant may be transdermal. Implants may include, for example, cardiovascular implants, orthopedic implants, contraception implants, cosmetic implants, prosthetic limbs, and ocular implants. In some cases, an implant may be a neural lace, and may be implanted into the head cavity, and may be in or near the brain. In some cases, a composition described herein may provide benefits for neuroplasticity and may positively alter the ability of the brain to change over time. Compositions formulated for inhalation can be packaged in an inhaler or nebulizer. An inhaler can be designed to dispense 0.25, 0.5, or 1 unit dose per inhalation. An inhaler can have a canister that holds the subject composition formulated for inhalation, a metering valve that allows for a metered quantity of the formulation to be dispensed with each actuation, and an actuator or mouthpiece that allows for the device to be operated and direct the subject composition into the subject's lungs. The formulated composition can include a liquefied gas propellant and possibly stabilizing excipients. The actuator can have a mating discharge nozzle that connects to the canister and a dust cap to prevent contamination of the actuator. Upon actuation, the subject composition can be volatized, which results in the formation of droplets of the subject composition. The droplets can rapidly evaporate resulting in micrometer-sized particles that may be then inhaled by the subject.

Compositions of the present disclosure suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, including liquid dosage forms (e.g., a suspension or slurry), and oral solid dosage forms (e.g., a tablet or bulk powder). Oral dosage forms can be formulated as tablets, pills, dragees, capsules, emulsions, lipophilic and hydrophilic suspensions, liquids, gels, syrups, slurries, suspensions, for oral ingestion by an individual, patient, or subject to be treated. Such dosage forms can be prepared by any of the methods of formulation. For example, the active ingredients can be brought into association with a carrier, which constitutes one or more necessary ingredients. Capsules suitable for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, in some cases, stabilizers. The composition for oral use may be obtained by mixing a composition comprising a solid excipient, in some cases grinding a resulting mixture, and processing the mixture of granules, after adding suitable auxiliaries, if desired, to obtain tablets or dragee cores. Excipients may be fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). Compositions that are prepared may be prepared uniformly and intimately admixing the active ingredient with liquid carriers or finely divided solid carriers or both, and then, if necessary, shaping the product into the desired presentation. For example, a tablet can be prepared by compression or molding, in some cases with one or more accessory ingredients. Compressed tablets can be prepared by compressing in a suitable machine the active ingredient in a free-flowing form, such as powder or granules, in some cases mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The liquid forms, in which the formulations disclosed herein can be incorporated for administration orally or by injection, include aqueous solution, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin.

Liquid preparations for oral administration can take the form of, for example, solutions, syrups or suspensions, or they can be presented as a dry product for reconstitution with water or other suitable vehicles before use. Such liquid preparations can be prepared by conventional approaches with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

This disclosure further encompasses anhydrous compositions and dosage forms comprising an active ingredient, since water can facilitate the degradation of some compounds. For example, water can be added (e.g., 5%) to simulate long-term storage in order to determine characteristics such as shelf-life or the stability of formulations over time. Anhydrous compositions and dosage forms of the present disclosure can be prepared using anhydrous or low moisture containing ingredients and low moisture or low humidity conditions. Compositions and dosage forms of the present disclosure which contain lactose can be made anhydrous if substantial contact with moisture and/or humidity during manufacturing, packaging, and/or storage is expected. An anhydrous composition can be prepared and stored such that its anhydrous nature is maintained. Accordingly, anhydrous compositions can be packaged using materials that prevent exposure to water such that they can be included in suitable formulary kits. Examples of suitable packaging include, but are not limited to, hermetically sealed foils, plastic, unit dose containers, blister packs, and strip packs.

An ingredient described herein can be combined in an intimate admixture with a pharmaceutical carrier according to conventional pharmaceutical compounding techniques. The carrier can take a wide variety of forms depending on the form of preparation desired for administration. In preparing the compositions for an oral dosage form, pharmaceutical media can be employed as carriers, such as, for example, water, glycols, oils, alcohols, flavoring agents, preservatives, coloring agents, in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, with or without employing the use of lactose. For example, suitable carriers include powders, capsules, and tablets, with the solid oral preparations. If desired, tablets can be coated by standard aqueous or nonaqueous techniques.

Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Binders suitable for use in dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Lubricants which can be used to form compositions and dosage forms of the present disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant may be added, such as, for example, in an amount of less than about 1 weight percent of the composition.

Lubricants can be also be used in conjunction with tissue barriers which include, but are not limited to, polysaccharides, polyglycans, seprafilm, interceed and hyaluronic acid.

Disintegrants can be used in the compositions of the present disclosure to provide tablets that disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active ingredient(s) can be used to form the dosage forms of the compounds disclosed herein. The amount of disintegrant used can vary based upon the type of formulation and mode of administration, and can be readily discernible to those of ordinary skill in the art. About 0.5 to about 15 weight percent of disintegrant, or about 1 to about 5 weight percent of disintegrant, can be used in the pharmaceutical composition. Disintegrants that can be used to form compositions and dosage forms of the present disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Examples of suitable fillers for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In some cases, aqueous suspensions and/or elixirs may be desired for oral administration. In such cases, the active ingredient therein may be combined with various sweetening or flavoring agents, coloring matter or dyes and, if desired, emulsifying and/or suspending agents, together with diluents such as water, ethanol, propylene glycol, glycerin or various combinations thereof.

The tablets can be uncoated or coated to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

In one embodiment, the composition can include a solubilizer to ensure good solubilization and/or dissolution of the compound of the present disclosure and to minimize precipitation of the compound of the present disclosure. This can be especially important for compositions for non-oral use, e.g., compositions for injection. A solubilizer can also be added to increase the solubility of the hydrophilic drug and/or other components, such as surfactants, or to maintain the composition as a stable or homogeneous solution or dispersion.

The composition can further include one or more pharmaceutically acceptable additives or pharmaceutically acceptable excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof. A non-exhaustive list of examples of excipients includes monoglycerides, magnesium stearate, modified food starch, gelatin, microcrystalline cellulose, glycerin, stearic acid, silica, yellow beeswax, lecithin, hydroxypropylcellulose, croscarmellose sodium, and crospovidone.

In some embodiments, a compound or composition described herein may be formulated or administered in combination with another active ingredient or ingredients. In some cases, a cannabinoid composition may be administered with psychedelic compounds for therapeutic enhancement. The therapeutic enhancement may be via optimization of the endocannabinoid system, neuroplasticity, neural trimming, anti-psychotic effects, anxiety effects, enhanced neurogenesis, or a combination thereof.

In some cases, a cannabinoid composition as described herein may be used in combination with psychedelic compounds, such as 3,4-methylenedioxymethamphetamine (MDMA), psilocybin, lysergic acid diethylamide (LSD). In some cases, a cannabinoid composition may be used in combination with psychedelic assisted therapeutic programs, and may assist in overall efficacy.

The compositions described herein can also be formulated as extended-release, slow-release, sustained-release or time-release such that one or more components are released over time. Compositions of the present disclosure may have half-lives of at least about 1 minute, 10 minutes, 30 minutes, 1 hours, 2 hours, 3 hours, 4 hours, 5 hours, 6 hours, 12 hours, 1 day, 2 days, 3 days, 4 days, 5 days, 6 days, 1 week, 2 weeks, 3 weeks, 4 weeks, or more. Delayed release can be achieved by formulating the one or more components in a matrix of a variety of materials or by microencapsulation (e.g., microencapsulation in a material that has a predetermined rate of degradation, or a material with pores with pore sizes that permit controllable release). The compositions can be formulated to release one or more components over a time period of 1, 4, 6, 8, 12, 16, 20, 24, 36, or 48 hours. The release of the one or more components can be at a constant or changing rate.

A composition described herein can be formulated in as matrix pellets in which particles of the subject composition are embedded in a matrix of water-insoluble plastic and which are enclosed by a membrane of water-insoluble plastic containing embedded particles of lactose, produces and maintains plasma levels of the subject composition within the targeted therapeutic range. A composition can be formulated as a sustained or controlled release capsule or tablet. A sustained or controlled release tablet may be formed by coating core granules composed mainly of the subject composition with a layer of a coating film composed of a hydrophobic material and a plastic excipient and, in some cases, containing an enteric polymer material, to form coated granules and then by compressing the coated granules together with a disintegrating excipient. Such sustained or controlled release capsule or tablet may release the composition in a substantially sustained or controlled manner over a given period of time, such as a substantially constant rate of release over a period of at least 0.1 hour, 0.5 hours, 1 hour, 2 hours, 3 hours, 4 hours, 5 hours, 6 hours, 12 hours, 24 hours, or more. Such sustained or controlled release capsule or tablet may permit at least about 10%, 20%, 30%, 40%, 50%, 60%, 70%, 80%, 90%, 95%, or greater of the composition to be released over a period of at least 0.1 hour, 0.5 hours, 1 hour, 2 hours, 3 hours, 4 hours, 5 hours, 6 hours, 12 hours, 24 hours, or more.

Using the controlled release dosage forms provided herein, the one or more cofactors can be released in its dosage form at a slower rate than observed for an immediate release formulation of the same quantity of components. The rate of change in the biological sample may be measured as the change in concentration over a defined time period from administration to maximum concentration for an controlled release formulation is less than about 80%, 70%, 60%, 50%, 40%, 30%, 20%, or 10% of the rate of the immediate release formulation. The rate of change in concentration over time may be less than about 80%, 70%, 60%, 50%, 40%, 30%, 20%, or 10% of the rate for the immediate release formulation.

The rate of change of concentration over time may be reduced by increasing the time to maximum concentration in a relatively proportional manner. For example, a two-fold increase in the time to maximum concentration can reduce the rate of change in concentration by approximately a factor of 2. As a result, the one or more cofactors can be provided so that it reaches its maximum concentration at a rate that is significantly reduced over an immediate release dosage form. The compositions of the present disclosure can be formulated to provide a shift in maximum concentration by 24 hours, 16 hours, 8 hours, 4 hours, 2 hours, or at least 1 hour. The associated reduction in rate of change in concentration can be by a factor of about 0.05, 0.10, 0.25, 0.5 or at least 0.8. This may be accomplished by releasing less than about 30%, 50%, 75%, 90%, or 95% of the one or more cofactors into the circulation within one hour of such administration.

The controlled release formulations may exhibit plasma concentration curves having initial (e.g., from 2 hours after administration to 4 hours after administration) slopes less than 75%, 50%, 40%, 30%, 20% or 10% of those for an immediate release formulation of the same dosage of the same cofactor.

The rate of release of the cofactor as measured in dissolution studies may be less than about 80%, 70%, 60%, 50%, 40%, 30%, 20%, or 10% of the rate for an immediate release formulation of the same cofactor over the first 1, 2, 4, 6, 8, 10, or 12 hours.

The controlled release formulations provided herein can adopt a variety of formats. The formulation may be in an oral dosage form, including liquid dosage forms (e.g., a suspension or slurry), and oral solid dosage forms (e.g., a tablet or bulk powder), such as, but not limited to those, those described herein.

Tablets or pills can also be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings such materials including a number of polymeric acids and mixtures of polymeric acids with such materials as shellac, cetyl alcohol and cellulose acetate. A formulation comprising a plurality of cofactors can have different cofactors released at different rates or at different times. For example, there can be additional layers of cofactors interspersed with enteric layers.

The compositions can be formulated in a food composition. For example, the compositions can be a beverage or other liquids, solid food, semi-solid food, with or without a food carrier. For example, the compositions can include a black tea supplemented with any of the compositions described herein. The composition can be a dairy product supplemented any of the compositions described herein. The compositions can be formulated in a food composition. For example, the compositions can comprise a beverage, solid food, semi-solid food, or a food carrier.

Liquid food carriers, such as in the form of beverages, such as supplemented juices, coffees, teas, sodas, and flavored waters can be used. For example, the beverage can comprise the formulation as well as a liquid component, such as various deodorant or natural carbohydrates present in conventional beverages. Examples of natural carbohydrates include, but are not limited to, monosaccharides such as, glucose and fructose; disaccharides such as maltose and sucrose; conventional sugars, such as dextrin and cyclodextrin; and sugar alcohols, such as xylitol and erythritol. Natural deodorant such as taumatin, stevia extract, levaudioside A, glycyrrhizin, and synthetic deodorant such as saccharin and aspartame can also be used. Agents such as flavoring agents, coloring agents, and others can also be used. For example, pectic acid and the salt thereof, alginic acid and the salt thereof, organic acid, protective colloidal adhesive, pH controlling agent, stabilizer, a preservative, glycerin, alcohol, or carbonizing agents can also be used. Fruit and vegetables can also be used in preparing foods or beverages comprising the formulations discussed herein.

Alternatively, the compositions can be a snack bar supplemented with any of the compositions described herein. For example, the snack bar can be a chocolate bar, a granola bar, or a trail mix bar. In yet another embodiment, the present dietary supplement or food compositions are formulated to have suitable and desirable taste, texture, and viscosity for consumption. Any suitable food carrier can be used in the present food compositions. Food carriers of the present disclosure include practically any food product. Examples of such food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables. In an embodiment, food carriers employed herein can mask the undesirable taste (e.g., bitterness). Where desired, the food composition presented herein exhibit more desirable textures and aromas than that of any of the components described herein. For example, liquid food carriers can be used according to the present disclosure to obtain the present food compositions in the form of beverages, such as supplemented juices, coffees, teas. Solid food carriers can be used according to the present disclosure to obtain the present food compositions in the form of meal replacements, such as supplemented snack bars, pasta, breads. Alternatively, semi-solid food carriers can be used according to the present disclosure to obtain the present food compositions in the form of gums, or chewy candies or snacks.

### Methods

In some embodiments, the present disclosure provides a method of converting a carboxylated cannabinoid to a decarboxylated cannabinoid. Such conversion can include removing a carboxylic acid group from the carboxylated cannabinoid.

In some embodiments, the present disclosure provides a method of supplementing a cannabinoid compound and a terpenoid to subject in need thereof, comprising administering a unit dose of a composition described herein.

The method of converting a carboxylated cannabinoid to a decarboxylated cannabinoid may be a chemical reaction. The chemical reaction conditions may comprise a catalyst, such as an acid, to facilitate conversion of a carboxylated cannabinoid to a decarboxylated cannabinoid. An acid used may be a weak acid. The acid may have a pKa that is at most about 10, 9, 8, 7, 6, 5, 4, 3, 2, 1, 0, −1, −2, −3, −4, −5, −6, −7, −8, −9, −10, or less. The acid may have a pKa that is at least about −10, −9, −8, −7, −6, −5, −4, −3, −2, −1, 0, 1, 2, 3, 4, 5, 6, 7, 8, 9, or more. The acid may have a pKa that is from about −10 to about 10, from about −7 to about 7, from about −3 to about 7, from about −1 to about 7, from about 3 to about 7, or from about 4 to about 6, or from about 5 to 7. In some embodiments, the acid has a pKa of at most about 20, 15, 10, or 5. In some embodiments, the acid has a pKa of at least about −10, −5, 0, 5, or more. The acid may be a weak acid. The acid may be a strong acid. The acid may be an organic acid. The acid may comprise a carboxylic acid moiety. The acid may have a molecular weight of less than about 500 daltons, 400 daltons, 300 daltons, 200 daltons, 100 daltons, 90 daltons, 80 daltons, 70 daltons, 60 daltons, 50 daltons, 40 daltons, or less.

The amount of acid used to convert a carboxylated cannabinoid (e.g. tetrahydrocannabinolic acid) to a decarboxylated cannabinoid (e.g. Δ⁹ tetrahydrocannabinol) may be selected such that the acid is catalytic (i.e., catalyzes the conversion of the carboxylated cannabinoid to a decarboxylated cannabinoid). The amount of acid may be at least about 0.01 grams (g), 0.1 g, 0.5 g, 1 g, 2 g, 5 g, 10 g, 20 g, 30 g, 40 g, 50 g, 60 g, 70 g, 80 g, 90 g, 100 g, or more.

The acid that may be used to convert a carboxylated cannabinoid to a decarboxylated cannabinoid may include one or more members selected from the group consisting of: lactic acid, citric acid, malic acid, acetic acid, benzoic acid, butyric acid, ascorbic acid, lactic acid, tartric acid, tannic acid, and oxalic acid. An acid may be an edible acid. The acid may be naturally occurring, and may be isolated from a natural source or may be produced synthetically. Depending on which acid is used, the acid may be diluted (e.g., with water) to provide an acidic solution having a pKa that may be suitable for conversion of a carboxylated cannabinoid to a decarboxylated cannabinoid.

The conversion may be performed in a laboratory, a production facility, in a home, or in a doctor's office, and may be performed by a technician, a doctor, or by a buyer or user of a kit described herein.

Conversion of a carboxylated cannabinoid to a decarboxylated cannabinoid may be under temperature conditions of at most 500° C., 400° C., 300° C., 200° C., 100° C., 75° C., 50° C., 40° C., or 30° C. The conversion may not require an external heat source or additional heating to perform the reaction of a carboxylated cannabinoid to a decarboxylated cannabinoid.

Carboxylated cannabinoid may be converted to decarboxylated by contacting a carboxylated cannabinoid with an acid. This may be performed by blending, mixing, stirring, or any combination thereof. The conversion may take place in a reaction vessel, such as, for example, a bowl, a round bottom flask, a reactor, a batch reactor, a plug flow reactor, catalytic reactor, a semi-batch reactor, or a household container. The reaction vessel may be configured to hold a reaction mixture. The reaction mixture may comprise an acid and part of a cannabis plant. The part of a cannabis plant may contain tetrahydrocannabinolic acid.

A reaction vessel may hold at least about 1 grams (g) of starting material, 2 g, 5 g, 10 g, 20 g, 30 g, 40 g, 50 g, 60 g, 70 g, 80 g, 90 g, 100 g, or more of starting material. The starting material may be part of a cannabis plant. The cannabis plant may contain carboxylated cannabinoids. The part of a cannabis plant may contain at least about 1 g, 2 g, 5 g, 10 g, 20 g, 30 g, 40 g, 50 g, 60 g, 70 g, 80 g, 90 g, 100 g or more of tetrahydrocannabinolic acid. A reaction vessel may hold at least about 1 milliliter (mL) of volume, 2 mL, 5 mL, 10 mL, 20 mL, 30 mL, 40 mL, 50 mL, 60 mL, 70 mL, 80 mL, 90 mL, 100 mL, 1 liter (L), 2 L, 5 L, 10 L, 50 L, 100 L, 1000 L, or more. A reaction vessel may be configured to provide production of at least about 1 grams (g) of a decarboxylated cannabinoid compound, 2 g, 5 g, 10 g, 20 g, 30 g, 40 g, 50 g, 60 g, 70 g, 80 g, 90 g, 100 g, or more of a decarboxylated cannabinoid compound.

A reaction mixture may undergo separation after the reaction is completed or near completion. Separation of the desired decarboxylated cannabinoids from the remainder of the reaction mixture, such as solvent, catalyst, plant material, or unconverted starting material, may include filtration, extraction, centrifugation, solubilization, concentration, washing, electrolysis, adsorption, purification, chromatography, fractionation, crystallization, or a combination thereof. In some cases, the desired decarboxylated cannabinoid (e.g. Δ⁹ tetrahydrocannabinol), is separated from the reaction mixture via 1, 2, 3, 4, 5 or more separation steps or procedures.

A trace amount of acid may be present in the reaction mixture after the reaction of a carboxylated cannabinoid to a decarboxylated cannabinoid. A trace amount of acid may be present after isolation of the desired compound from the reaction mixture. The separated or isolated decarboxylated cannabinoid may be the desired final product that may be used in manufacturing processes for delivery to a user. The isolated decarboxylated cannabinoid may comprise a trace amount of acid. A trace amount of acid may be less than about 10,000 parts per millions (ppm), 1000 ppm, 100 ppm, 10 ppm, or 1 ppm.

### Indications

Terpenoids may have a beneficial effect on a subject. Terpenoids may be formulated into a composition described herein and given to a subject as a nutritional supplement or a dietary supplement. A composition may provide nutrients or compounds that may otherwise not be consumed in sufficient quantities in a normal diet. A composition may contain compounds that may be beneficial to the health, physical wellbeing, and emotional wellbeing of a subject. For example, a composition may be used as a boost of energy.

A composition of the current disclosure may be used to treat or decrease the symptoms of nausea or vomiting. A subject who is administered a unit dose of a composition may observe a decrease in symptoms related to nausea or vomiting.

A composition of the current disclosure may be used to treat an eating disorder, such as anorexia, cachexia, bulimia nervosa, rumination disorder, avoidant or restrictive food intake disorder.

A composition of the current disclosure may be used as a sleep aid or to help with symptoms of insomnia. A composition may help a subject relax, fall asleep faster, improve sleep quantity, or improve sleep quality.

A composition of the current disclosure may be used to mediate, limit, and reverse the effects of oxidative damage or oxidative stress. The imbalance of reactive oxygen species within a subject may be corrected or mediated with administration of a composition described herein.

Oxidative stress may occur prior to, during, and/or after surgery. The oxidative stress may be due to anesthesia used during the surgery, the surgical trauma, the psychological stress associated with surgery, or a combination thereof. A composition may be administered to a subject prior to, during, or after surgery. A composition may be administered to relieve traumatic shock that may be caused by surgery or injury.

Oxidative stress may be caused by a physical stress factor or an emotional stress factor. A composition of the current disclosure may be used to treat post-traumatic stress disorder (PTSD). Some symptoms of PTSD that may be relieved by a composition include: flashbacks, bad dreams, frightening thoughts, avoidance of certain thoughts or feelings, being easily startled, feeling tense, having difficulty sleeping, cognition or mood symptoms such as trouble remember features of the traumatic event, distorted feelings such as guilt or blame, and loss of interest in enjoyable activities.

A composition may be administered to prevent or limit the severity of developing post-traumatic stress disorder. The composition may be administered after physical or emotional stress, such as, for example, after a physical sport, a contact sport, a physical combat, a physical confrontation, a military drill or exercise, and military combat.

A composition of the current disclosure may be used to treat, alleviate, or cease the symptoms of addiction, addicted behavior, physical dependence, or psychological dependence. Addiction may be characterized by compulsive engagement in stimuli. Addiction may be rated based on a severity index, such as the addiction severity index (ASI). The ASI severity ratings may be based on a 10 point scale, from 0-9. A rating of 0-1 may be classified as no real problem, treatment not indicated. A rating of 2-3 may be classified as a light problem, treatment may not be indicated. A rating of 4-5 may be classified as a moderate problem, and some treatment may be indicated. A rating of 6-7 may be considerable a problem, and treatment may be necessary. A rating of 8-9 may be considered an extreme problem, and treatment may be absolutely necessary.

Examples of drug and behavioral addictions include, but are not limited to, alcoholism, cocaine addiction, smoking addiction, nicotine addiction, opiate addiction, food addiction, amphetamine addiction, and gambling addiction.

A composition described herein may be used to alleviate smoking addiction. A composition may be used as part of a smoking cessation program, where a subject is administered tobacco infused with cannabinoids. A composition may also be administered via water soluble methods, to allow for membrane absorption for natural and gradual decrease in addiction. Additionally, a composition described herein may contribute to anti-anxiety effects. For example, a composition administered in water soluble form may provide rapid anti-anxiety effects to curb addition via mucosal membrane absorption. A cannabinoid composition may be part of program to decrease tobacco usage over time.

In some cases, a cannabinoid compound may have multiple bell curves of efficacy. The bell curves of efficacy may change or modulate on a daily basis depending on a number of factors of the subject, including oxidative stress.

A composition of the current disclosure may be administered to a subject during and/or after treatment. A subject may observe a decrease symptoms or a decrease in severity rating according to a severity index scale (e.g. the ASI severity index).

A composition may be used to treat cancer or a tumor. Cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. A composition described herein may be used to treat cervical cancer.

A composition described herein may be used to treat prostate cancer. A subject may be evaluated based on a level of prostate-specific antigen, or PSA, a protein produced by prostate gland cells. Elevated blood levels of PSA may be associated with subjects with prostate cancer.

A composition may be administered to a subject that has been diagnosed with prostate cancer. In some cases, a composition may be administered to a subject with a PSA greater than about 1 nanograms per milliliter (ng/mL), 2 ng/mL, 3 ng/mL, 4 ng/m L, 5 ng/mL, or 6 ng/mL, or higher. Administration with a composition may have a dosing holiday after a subject's level of PSA is below a certain threshold. In some cases, a dosing holiday may begin after PSA drops below about 20 nanograms per milliliter (ng/mL), 10 ng/mL, 5 ng/mL, 4 ng/mL, 3 ng/mL, 2 ng/mL, or 1 ng/mL. A dosing holiday of a composition may be substituted with another compound or composition. For example, an amount of delta-9-tetrahydrocannabinol (THC) may be administered during a dosing holiday of a composition described herein. The amount of THC administered may be at most about 50 mg/kg, 40 mg/kg, 30 mg/kg, 20 mg/kg, 10 mg/kg, 5 mg/kg, 4 mg/kg, 3 mg/kg, 2 mg/kg, 1 mg/kg, or less.

A composition of the current disclosure may be used to treat an eating disorder or a weight disorder, such as, for example, anorexia and cachexia. Subjects may observe an increase in appetite after at least a unit dose of a composition of the present disclosure. In some cases, Δ⁹ tetrahydrocannabinol in a composition may result in an appetite enhancing effect with a unit dose of at least 1 mg, 2 mg, 3 mg, 4 mg, 5 mg, 10 mg, 15 mg, or 20 mg. An increase in appetite may continue for an extended period of time, such as at least 1 day, 2 days, 5 days, 7 days, 1 week, 2 weeks, 1 month, 3 months, 6 months, or 12 months after the last unit dose of a composition is taken.

A composition of the current disclosure may be used to treat a muscle related disorder or a movement disorder, such as, for example, spasticity, tremor, ataxia, bladder control, Tourette's syndrome, dystonia, Parkinson's disease, Huntington disease, and tardive dyskinesia. Spasticity may have been caused by pain, bone or join deformities, or accidents or injury to the spinal cord.

A composition of the current disclosure may be used to treat pain. The pain may be an acute pain. The pain may be chronic pain. The pain may be associated with a disease. Pain in a subject may be neuropathic pain, menstrual pain, chronic headaches, or back pain. Pain may be due to a disease or a disorder, or may be caused by injury. Pain may be caused by a disease such as cancer, chronic bowel inflammation, neuralgias, damaged nerves, diabetes, multiple sclerosis, an infection, or old age.

Pain can be nociceptive pain (i.e., pain caused by tissue damage), neuropathic pain or psychogenic pain. The pain may be caused by or associated with a disease (e.g., cancer, arthritis, diabetes). Alternatively, the pain is caused by injury (e.g., sports injury, trauma). Non-limiting examples of pain that may be amenable to treatment with the compositions and methods herein include: neuropathic pain including peripheral neuropathy, diabetic neuropathy, post herpetic neuralgia, trigeminal neuralgia, back pain, neuropathy associated with cancer, neuropathy associated with HIV/AIDS, phantom limb pain, carpal tunnel syndrome, central post-stroke pain, pain associated with chronic alcoholism, hypothyroidism, uremia, pain associated with multiple sclerosis, pain associated with spinal cord injury, pain associated with Parkinson's disease, epilepsy, osteoarthritic pain, rheumatoid arthritic pain, visceral pain, and pain associated with vitamin deficiency; and nociceptive pain including pain associated with central nervous system trauma, strains/sprains, and burns; myocardial infarction, acute pancreatitis, post-operative pain, posttraumatic pain, renal colic, pain associated with cancer, pain associated with fibromyalgia, pain associated with carpal tunnel syndrome, and back pain.

The compositions and methods described herein may be utilized to ameliorate a level of pain in a subject. A level of pain in a subject may be ameliorated by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or 100%. A level of pain in a subject can be assessed by a variety of methods. A level of pain may be assessed by self-reporting (i.e., a human subject expresses a verbal report of the level of pain he/she is experiencing). A level of pain may be assessed by behavioral indicators of pain, for example, facial expressions, limb movements, vocalization, restlessness and guarding. These types of assessments may be useful for example when a subject is unable to self-report (e.g., an infant, an unconscious subject, a non-human subject). A level of pain may be assessed after treatment with a composition of the present disclosure as compared to the level of pain the subject was experiencing prior to treatment with the composition.

A composition of the current disclosure may be used to reduce intraocular pressure or fluid pressure in the eye, and may be used to treat a number of diseases associated with abnormal intraocular pressure, including, but not limited to, glaucoma, iritis, retinal detachment. A composition may decrease intraocular pressure by 5%, 10%, 20%, 30%, 40%, 50%, or more.

The methods and compositions of the present disclosure may be utilized to treat epilepsy. Compositions described herein may be used to prevent or control epileptic seizures. Epileptic seizures may be classified as tonic-clonic, tonic, clonic, myoclonic, absence or atonic seizures. The compositions and methods herein may prevent or reduce the number of epileptic seizures experienced by a subject by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or higher.

The methods and compositions of the present disclosure may be utilized to relieve the symptoms of an inflammatory disease of the airways of the lungs, or asthma. The compositions may reduce the severity of asthma symptoms, or change the severity classification, such as from severe persistent, to moderate persistent, to mild persistent, to intermittent.

A composition of the current disclosure may be used to relieve symptoms associated with withdrawal in dependency on alcohol and drugs, such as benzodiazepines and opiates. The composition may relieve withdrawal symptoms such as sleep disturbance, irritability, increased tension, anxiety, panic attacks, tremors, sweating, difficulty concentrating, confusion, memory loss, weight loss or weight gain, headaches, or muscular pains.

A composition of the current disclosure may be used to treat psychiatric disorders, including, but not limited to, sleep disorder, anxiety disorders, panic disorders, obsessive-compulsive disorder, bipolar disorder, depression, mood disorders, personality disorders, psychotic disorders, such as schizophrenia or delusional disorder. A composition may be used to treat a bipolar episode, wherein a symptom may include an unusual shift in mood, energy, activity level, and the inability to carry out day-to-day tasks.

A composition of the current disclosure may be used to treat autoimmune diseases or inflammation, such as, but not limited to, arthritis, lupus, vitiligo, anemia, psoriasis, scleroderma, inflammatory bowel diseases, and type 1 diabetes.

A composition may be used to treat pruritus, ADS (attention deficit syndrome), high blood pressure, tinnitus, chronic fatigue syndrome, and restless leg syndrome.

A composition may be used to treat or relieve the symptoms of the hiccups or synchronous diaphragmatic flutter (SDF). Hiccups may be classified as acute, chronic, persistent, or intractable. In some cases, a compound or composition may be used to treat or alleviate the symptoms of chronic hiccups.

A composition may be used to treat or alleviate the symptoms of menopause or pre-menopause. A composition may decrease the frequency and/or intensity of symptoms that include, for example, hot flashes, night sweats, pain during intercourse, increased anxiety or irritability, and the need to urinate more often.

A composition may be used to treat or sterilize wounds. A composition may be used in conjunction with citric acid, or may be formulated into one composition for use in sterilizing open wounds.

A composition described herein may be used with poison or venom treatment. The composition may be administered before, during, or after administration of a poison antidote or an antivenom. The composition may be administered after exposure to a toxin or poison and may be in the absence of an antidote. Administration for use with a poison antidote may be via injection, sublingual, oral, via nasal spray, or a transdermal patch. Without wishing to be bound by theory, the cannabinoid composition may help protect the tissue, nervous system, and/or assist with regulating overall homeostasis in the subject. The cannabinoid composition may help decrease oxidative stress, tissue damage, organ damage, or neural trauma. The composition may also enhance cellular protection, health, and overall homeostatic balance.

In some cases, a composition described herein may be used for treatment of shingles, chicken pox, measles, human papillomavirus (HPV), chronic obstructive pulmonary disease (COPD), emphysema, ilitigo, impetigo, pneumonia, listeria, Ebola, Addison's disease, Graves' disease, Sjögren's syndrome, Hashimoto's disease, autism, myasthenia gravis, Pernicious Anemia, or Celiac disease.

In some cases, a composition may be used to treat an autoimmune disease. In some cases, a composition may be used to treat Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, or Wegener's granulomatosis (or Granulomatosis with Polyangiitis (GPA)).

A composition comprising cannabinoids may be formulated in water soluble form. Administration of the composition in a water soluble form may allow for rapid membrane absorption. The composition may be added to a water supply, e.g., drinking water, for protection after a chemical agent or toxicity event or exposure.

A composition may be used to enhance neurogenesis, or the growth and development of nervous tissue in a subject. A composition may also enhance the overall performance of the nervous system, including the parasympathetic nervous system, the sympathetic nervous system, and enteric nervous system.

A composition may be used as a supplement to protect a telomere, a region of the end of a chromosome in a subject. Protection of a telomere may protect the chromosome from deterioration.

In some cases, a composition described herein may be used as a targeted endocannabinoid system (ECB) therapeutic. Two primary endocannabinoid receptors of the endocannabinoid system are CB1 and CB2.

In some cases, a composition of the current disclosure may be used in combination with epigenetics, or the study of heritable changes in gene function that may not involved changes in the DNA sequence, or functionally relevant changes to the genome that may not involve a change in the nucleotide sequence (e.g. DNA methylation, histone modification). In some cases, epigenetic mechanisms may include factors and processes such as development (e.g. in utero, childhood), environmental factors (e.g. environmental chemicals), drugs, pharmaceuticals, aging, and diet.

A composition may be administered or suggested based on epigenetic testing. In some cases, a composition described herein can modulate risk factors or improve disease conditions. In some cases, terpenes, such as those described herein, may be used to direct cannabinoids to specific CB receptor sites. Compounds disclosed herein may be used to prevent to mitigate risks or harm during or after epigenetic indication and may contribute to changing the expression.

In some cases, a composition could be used to treat the thyroid if a thyroid risk factor was apparent, then the same composition could be used to target a different region of the body using different terpenes if a new epigenetic expression appeared. In some cases, a composition may have rapid absorption in the body. If a composition has rapid absorption, the same formula may be given sequentially and may change the effects of the cannabinoids.

In some cases, a composition may be administered or suggested based on genetic testing.

Alternatively, a composition may be administered based on standard testing for targeted treatment protocols, wherein cannabinoids and terpenes in the composition may prevent and/or treat risk factors or disease states.

A subject may exhibit one or more symptoms. A symptom may be selected from pain, stress, nausea, vomiting, sleeplessness, anxiety, and appetite loss. A unit dose may be used to alleviate a symptom in a subject, and in some cases, by at least about 10%, 20%, 30%, 40%, 50%, 60%, 70%, 75%, 80%, 85%, 90%, 95%, or 99%. A composition may result in a decrease of the severity or quantity of symptoms by at least about 10%, 20%, 30%, 40%, 50%, 60%, 70%, 75%, 80%, 85%, 90%, 95%, or 99%.

A unit dose can be administered at least about 1, 2, 3, 4, 5, 6, 7, 8, 9, 10 or more times a daily. A subject can receive dosing for a period of about, less than about, or greater than about 1, 2, 3, 4, 5, 6, 7, 8, 9, 10, 11, 12, 13, 14 or more days, weeks or months. A unit dose can be a fraction of the daily dose, such as the daily dose divided by the number of unit doses to be administered per day. A unit dose can be a fraction of the daily dose that is the daily dose divided by the number of unit doses to be administered per day and further divided by the number of unit doses (e.g. tablets) per administration. The number of unit doses per administration can be at least 1, 2, 3, 4, 5, 6, 7, 8, 9, 10, or more. The number of doses per day can at least 1, 2, 3, 4, 5, 6, 7, 8, 9, 10, or more. The number of unit doses per day can be determined by dividing the daily dose by the unit dose, and can at least 1, 2, 3, 4, 5, 6, 7, 8, 9, 10, 11, 12, 13, 14, 15, 6, 17, 18, 19, 20, or more unit doses per day. For example, a unit dose can be about ½, ⅓, ¼, ⅕, ⅙, 1/7, ⅛, 1/9, or 1/10 of the recommended daily dose. A unit dose can be about one-third of the daily amount and administered to the subject three times daily. A unit dose can be about one-half of the daily amount and administered to the subject twice daily. A unit dose can be about one-fourth of the daily amount with two unit doses administered to the subject twice daily.

The length of the period of administration and/or the dosing amounts can be determined by a physician or any other type of clinician. The physician or clinician can observe the subject's response to the administered compositions and adjust the dosing based on the subject's performance. For example, dosing for subjects that show reduced effects in energy regulation can be increased to achieve desired results.

The components in the compositions can be administered together at the same time in the same route, or administered separately. The components in the compositions can also be administered subsequently. The components in the compositions can be administered at the same or different administration route.

Another aspect of the present disclosure provides for achieving desired effects in one or more subjects after administration of a combination composition described herein for a specified time period. For example, the beneficial effects of the compositions described herein can be observed after administration of the compositions to the subject for 1, 2, 3, 4, 6, 8, 10, 12, 24, or 52 weeks.

In some embodiment, the present disclosure also provides for methods of manufacturing the compositions described herein. The manufacture of a composition described herein may comprise mixing or combining two or more components.

The compositions can be combined or mixed with a pharmaceutically active or therapeutic agent, a carrier, and/or an excipient. Examples of such components are described herein. The combined compositions can be formed into a unit dosage as tablets, capsules, gel capsules, or slow-release tablets.

The composition may be prepared such that a solid composition containing a substantially homogeneous mixture of the one or more components is achieved, such that the one or more components are dispersed evenly throughout the composition so that the composition can be readily subdivided into equally effective unit dosage forms such as tablets, pills and capsules.

A unit dose of a composition may retain at least about 10%, 20%, 30%, 40%, 50%, 60%, 70%, 80%, 90%, or 95% of one or more cannabinoids after placement in a sealed container for a certain period of time, such as after 1 day, 7 days, 1 month, 6 months, 1 year, 2 years, 3 years, 4 years, 5 years, or more. A unit dose of a composition may have a shelf-life that is at least about 1 day, 7 days, 1 month, 6 months, 1 year, 2 years, 3 years, 4 years, 5 years, or more. Without wishing to be bound by theory, a trace amount of acid in a composition may contribute and enhance the shelf life of a composition.

A unit dose of a composition may be stored under conditions of at least about 25° C., 30° C., 40° C., 50° C., 60° C., 70° C., or more. A unit dose of a composition may be stored under a humidity level condition of at least about 10%, 20%, 30%, 40%, 50%, 60%, 70%, 80%, 90%, 95% or more humidity level without significant degradation of the cannabinoid.

A unit dose may be packaged into a container to be transferred to the user. A unit dose may be packaged in a tube, ajar, a box, a vial, a bag, a tray, a drum, a bottle, a syringe, or a can.

### Kits

The present disclosure also provides kits. The kits include one or more compositions described herein, in suitable packaging, and can further comprise written material that can include instructions for use, discussion of clinical studies, and listing of side effects. Such kits can also include information, such as scientific literature references, package insert materials, clinical trial results, and/or summaries of these, which indicate or establish the activities and/or advantages of the composition, and/or which describe dosing, administration, side effects, drug interactions, or other information useful to the health care provider. Such information can be based on the results of various studies, for example, studies using experimental animals involving in vivo models and studies based on human clinical trials. A kit can comprise one or more unit doses described herein. A kit may comprise at least about 1, 2, 3, 4, 5, 6, 7, 8, 9, 10, 11, 12, 13, 14, 15, 16, 17, 18, 19, 20, 30, 31, 60, 90, 120, 150, 180, 210, or more unit doses. A kit may comprise at most about 20, 15, 10, 5, 4, 3, 2, or 1 unit dose. Instructions for use can comprise dosing instructions, such as instructions to take 1, 2, 3, 4, 5, 6, 7, 8, 9, 10, or more unit doses 1, 2, 3, 4, 5, 6, 7, 8, 9, 10, or more times per day. For example, a kit can comprise a unit dose supplied as a tablet, with each tablet package separately, multiples of tablets packaged separately according to the number of unit doses per administration (e.g. pairs of tablets), or all tablets packaged together (e.g. in a bottle). As a further example, a kit can comprise a unit dose supplied as a bottled drink, the kit comprising 1, 2, 3, 4, 5, 6, 7, 8, 9, 10, 11, 12, 13, 14, 24, 28, 36, 48, 72, or more bottles.

Instructions may be provided in print form on a user interface of an electronic device of a user. The instructions may be provided, for example, on a graphical user interface or a web-based interface.

The kit can further contain another agent. The compound of the present disclosure and the agent may be provided or packaged as separate compositions in separate containers within the kit. The compound of the present disclosure and the agent may be provided or packaged as a single composition within a container in the kit. Suitable packaging and additional articles for use (e.g., measuring cup for liquid preparations, foil wrapping to minimize exposure to air) can be included in the kit. Kits described herein can be provided, marketed and/or promoted to health providers, including physicians, nurses, pharmacists, formulary officials. Kits can also be marketed directly to the consumer.

A kit can comprise a multi-day supply of unit dosages. The unit dosages can be any unit dosage described herein. The kit can comprise instructions directing the administration of the multi-day supply of unit dosages over a period of multiple days. The multi-day supply can be a one-month supply, a 30-day supply, or a multi-week supply. The multi-day supply can be a 90-day, 180-day, 3-month or 6-month supply. The kit can include packaged daily unit dosages, such as packages of 1, 2, 3, 4, or 5 unit dosages. The kit can be packaged with other dietary supplements, vitamins, and meal replacement bars, mixes, and beverages.

A kit may comprise starting materials that allows a user to perform the conversion of a carboxylated cannabinoid to a decarboxylated cannabinoid (e.g. Δ⁹ tetrahydrocannabinol).

A kit may comprise all the necessary starting materials so that a user may perform the conversion. The kit may comprise a carboxylated cannabinoid, an acid present to effect conversion of the carboxylated cannabinoid to a decarboxylated cannabinoid, a reaction vessel configured to hold a reaction mixture comprising the acid and the carboxylated cannabinoid, and instructions for performing the conversion utilizing the acid. The resulting decarboxylated cannabinoid that is formed from the conversion may be Δ⁹ tetrahydrocannabinol.

Alternatively, a kit may comprise some of the necessary starting materials so that a user may perform the conversion. A user may supplement the kit with his or her own supply of carboxylated cannabinoid. The kit may comprise an acid present to effect conversion of the carboxylated cannabinoid to a decarboxylated cannabinoid, a reaction vessel configured to hold a reaction mixture comprising the acid and the carboxylated cannabinoid, and instructions for performing the conversion utilizing the acid. The carboxylated cannabinoid that the user supplies may be tetrahydrocannabinolic acid.

The acid in a kit may be present in at least about 0.01 grams (g), 0.1 g, 0.5 g, 1 g, 2 g, 5 g, 10 g, 20 g, 30 g, 40 g, 50 g, 60 g, 70 g, 80 g, 90 g, 100 g, or more.

The carboxylated cannabinoid (e.g. tetrahydrocannabinolic acid), if provided in a kit, may be present in at least about 1 grams (g), 2 g, 5 g, 10 g, 20 g, 30 g, 40 g, 50 g, 60 g, 70 g, 80 g, 90 g, 100 g, or more. The amount of decarboxylated cannabinoid formed from performing a reaction using a kit may be at least about 1 grams (g), 2 g, 5 g, 10 g, 20 g, 30 g, 40 g, 50 g, 60 g, 70 g, 80 g, 90 g, 100 g, or more.

### Computer Control Systems

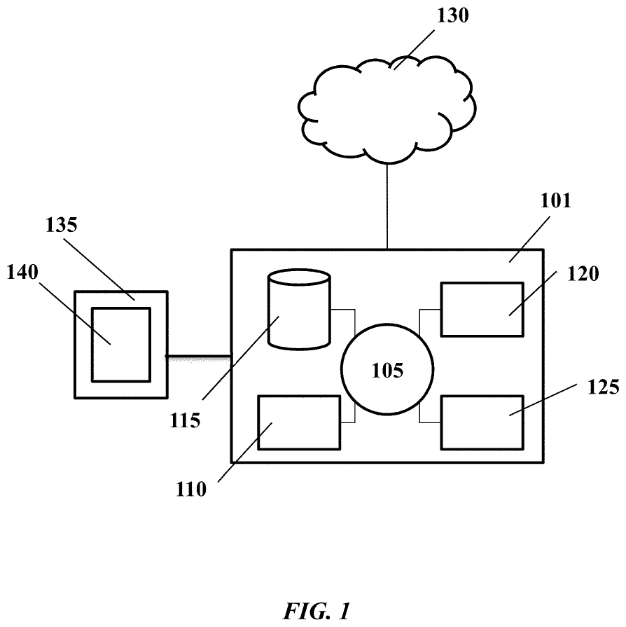

The present disclosure provides computer control systems that are programmed to implement methods of the disclosure. FIG. 1 shows a computer control system 101 that is programmed or otherwise configured to produce a composition comprising a mixture of carboxylated cannabinoids and decarboxylated cannabinoids, or provide instructions for using or generating compositions of the present disclosure. The computer control system 101 can regulate various aspects of the methods of the present disclosure, such as, for example, methods of producing the decarboxylated cannabinoids, including, but not limited to, the movements of the reaction vessel and stirrer, packaging of a unit dose of a composition, and printing instructions for use of a composition. The computer control system 101 can be implemented on an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer control system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer control system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user (e.g., a user controlling the manufacture of a three-dimensional object). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### EXAMPLES

### Example 1: Treatment of Pain Relief for a Subject with Chronic Pain with a Composition

Subjects (e.g., patients) that are suffering from chronic pain have their pain levels assessed and evaluated by a treating physician or other pain management expert. Such subjects are then prescribed 10 mg/day of a composition comprising cannabinoids and one or more terpenoids. The subjects are then evaluated again after two weeks to determine if symptoms have improved. After evaluation, the dosage is increased, decreased, or kept the same depending on the change in the level of pain. The treatment is maintained for as long as necessary to affect a stable resolution of the symptoms of chronic pain.

### Example 2: Treatment of a Subject that has been Diagnosed with Alzheimer's Disease

Subjects that are diagnosed clinically with Alzheimer's disease are evaluated for common symptoms such as memory loss and confusion.

Subjects are prescribed 10 mg/day of a composition, and then evaluated again after two weeks to determine if symptoms have worsened. After evaluation, the dosage is increased, decreased, or kept the same depending on the change in the symptoms of inattention and hyperactivity. The treatment is maintained for as long as necessary to affect a stable or desired level of the symptoms of Alzheimer's disease.

### Example 3: Synthesis of a Unit Dose of a Composition

A reaction vessel in an industrial laboratory is placed in an ice bath. The reaction vessel is charged with 100 grams of cannabis plant, 1 mL of acetic acid, and 5 L of water. The reaction is stirred as the ice bath is removed and the reaction is allowed to come to room temperature.

After the reaction is completed after 30 minutes, the reaction mixture is filtered and the water in the resulting solution is removed. The solid is then mixed with a filler, such as gelatin, and is packaged into a capsule as a unit dose.

The unit dose is then distributed and sold to users.

### Example 4: User Synthesis of a Unit Dose of a Composition Using a Kit

A kit is sold to a user. The kit comprises all the necessary starting materials so that the user may perform the conversion of a carboxylated cannabinoid to a decarboxylated cannabinoid. The kit comprises 2 grams of a cannabis plant, 1 grams of citric acid, a bowl, and instructions for performing the conversion utilizing the acid.

The user follows the instructions and places the cannabis plant and the citric acid into the bowl. The user then adds 1 cup of water, per the instructions provided in the kit. The mixture is stirred for 5 minutes to complete the reaction.

Methods and compositions provided herein may be combined with other methods and compositions, such as those described in PCT Patent Publication No. WO/2016/094810, which is entirely incorporated herein by reference.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1.-51. (canceled)

52. A unit dose comprising:
(i) a mixture of carboxylated cannabinoids and decarboxylated cannabinoids, and
(ii) one or more terpenoids,
wherein a wt/wt ratio of decarboxylated cannabinoids to carboxylated cannabinoids is greater than 0.5, and
wherein said unit dose is substantially free of a terpenoid degradation compound selected from the group consisting of: geraniol, geranyl isobutyrate, p-cymenene, p-cymene, p-mentha-1,5,8-triene, carvone, 3-methyl-6-(1-methylethylidene)-2-cyclohexen-1-one, 3-methyl-6-(1-methylethenyl)-2-cyclohexen-1-one, eucarvone, thymol, p-mentha-1 (7),8-dien-2-ol, perillyl alcohol, camphene, beta-myrcene, alpha-phellandrene, alpha-terpinene, gamma-terpinene, terpinolene, 4-hydroxy-2-methyl-2-cyclohexenone, p-cymenene, o-cymene, 3-caren-2-one, 3-caren-5-one, 3-carene oxide, 3-carene-2,5-dione, trans-2-hydroxy-3-caren-5-one, thymol, carvacrol, 1,4-cineole, eucalyptol, 3-(1-methylethyl)-6-oxo-2-heptenal, and 3,7-dimethyl6-oxo-2-octenal.

53. The unit dose of claim 52, further comprising at least 5 mg of decarboxylated cannabinoids.

54. The unit dose of claim 52, wherein said decarboxylated cannabinoids comprises Δ⁹ tetrahydrocannabinol.

55. The unit dose of claim 52, wherein said one or more terpenoids is selected from the group consisting of: myrcene, limonene, linalool, trans-ocimene, beta-pinene, alpha-pinene, beta-caryophyllene, delta-3-carene, trans-gamme-bisabolene, trans-alpha-farnesene, beta-fenchol, alpha-humulene, and guajol.

56. The unit dose of claim 52, wherein said unit dose is substantially free of terpenoid degradation compounds.

57. The unit dose of claim 52, further comprising an acid.

58. The unit dose of claim 52, further comprising a pharmaceutically acceptable excipient.

59. The unit dose of claim 58, wherein said pharmaceutically acceptable excipient is selected from the group consisting of: a binder, a filler, a plasticizer, a lubricant, an anti-foaming agent, a buffering agent, a polymer, an antioxidant, a preservative, a chelating agent, a flavorant, a colorant, an odorant, a suspending agent, and a combination thereof.

60. The unit dose of claim 52, wherein said unit dose is formulated for oral, topical, inhalation, intravenous, or intramuscular administration.

61. The unit dose of claim 52, wherein said unit dose is in a solid form.

62. The unit dose of claim 52, wherein said unit dose is in a liquid form.

63. The unit dose of claim 52, wherein said unit dose is a tablet, a chewable tablet, a capsule, a caplet, a pill, a granule, an emulsion, a gel, a spray, a plurality of beads encapsulated in a capsule, a powder, a suspension, a liquid, a semi-liquid, a semi-solid, a solution, a syrup, or a slurry.

64. The unit dose of claim 52, wherein said unit dose retains at least 80% of said carboxylated and decarboxylated cannabinoids after placement in a sealed container for 6 months at a temperature of about 25° C. and a relative humidity level of about 50%.

65. The unit dose of claim 52, wherein said unit dose is packaged into a container selected from the group consisting of a tube, ajar, a box, a vial, a bag, a tray, a drum, a bottle, a syringe, and a can.

66. A kit comprising a unit dose of claim 52 and instructions for supplementing said mixture of carboxylated cannabinoids and decarboxylated cannabinoids and one or more terpenoids to a subject in need thereof.

67. A mixture comprising:
(i) carboxylated cannabinoids and decarboxylated cannabinoids,
(ii) one or more terpenoids, and
(iii) an acid,
wherein a wt/wt ratio of decarboxylated cannabinoids to carboxylated cannabinoids is greater than 0.1, and
wherein said acid is present in an amount effective in converting at least a portion of carboxylated cannabinoids to decarboxylated cannabinoids.

68. The mixture of claim 67, wherein said mixture comprises at least 0.05 mol of said decarboxylated cannabinoids.

69. The mixture of claim 67, wherein said decarboxylated cannabinoids comprise Δ⁹ tetrahydrocannabinol.

70. The mixture of claim 69, wherein a wt/wt ratio of Δ⁹ tetrahydrocannabinol to tetrahydrocannabinolic acid is greater than about 0.1.

71. The mixture of claim 67, wherein said mixture is substantially free of terpenoid degradation compounds.
